(19) United States
(12) Patent Application Publication
(10) Pub. No.: US 2024/0226511 A9
(43) Pub. Date: Jul. 11, 2024

(54) METHOD OF FORMING A BALLOON

(71) Applicant: Medtronic, Inc., Minneapolis, MN (US)

(72) Inventors: Thomas A. Wong, Little Canada, MN (US); Jonathan E. Baxter, Fridley, MN (US); Kevin R. Seifert, Forest Lake, MN (US)

(21) Appl. No.: 17/972,462

(22) Filed: Oct. 24, 2022

(51) Int. Cl. A61M 25/10; C08L 27/18

(52) U.S. Cl. CPC A61M 25/1029; C08L 27/18; A61M 2025/1031

(57) ABSTRACT

Medical devices including a balloon and methods of making medical devices including a balloon are provided. The medical device includes a tubular element, a balloon, and a barrier between the tubular element and the balloon. When air is pushed into one end of the medical device, the balloon may be inflated. The method includes applying a barrier to a tubular element, applying an elastomer solution to the barrier and at least a part of the tubular element to form a coating, and curing the coating to form the balloon.

### FIELD

The present disclosure is related to medical devices including balloons and methods of forming medical devices including balloons. Specifically, this disclosure relates to tubular medical devices including elastic balloons for use in a body.

### SUMMARY

The present disclosure relates to a medical device including a tubular element comprising a tubular wall with a distal end and a proximal end, the tubular element comprising an inner lumen and one or more holes through the tubular wall between the distal and proximal ends, a balloon extending diametrically around the tubular wall, wherein the balloon is bonded to at least a portion of the tubular wall, and wherein at least a portion of the balloon is not bonded to the tubular wall, and a barrier between the tubular wall and the balloon, the barrier covering the one or more holes and being permeable to air.

According to an embodiment, one axial end of the tubular element is sealed. The tubular element may include more than one hole through the tubular wall. Additionally or alternatively, the tubular wall includes polymer, glass, or metal.

According to an embodiment, the balloon includes an elastomer. The elastomer may include silicone, natural rubber, neoprene, nitrile, or a combination thereof. The balloon may be bonded to the tubular wall adjacent to both axial ends of the barrier.

According to an embodiment, the barrier has a density of 1.0 g/cm³ or less. Additionally or alternatively, the barrier has a wall thickness of 0.2 mm or less. In one or more embodiments, the barrier includes polytetrafluoroethylene (PTFE), such as expanded PTFE (ePTFE).

In another aspect, the present disclosure relates to a method of forming a medical device comprising a balloon, the method comprising including applying a barrier to at least a portion of a tubular element, the tubular element comprising a tubular wall having a distal end and a proximal end and one or more holes through the tubular wall between the distal and proximal ends, the barrier covering the one or more holes and being permeable to air, applying an elastomer solution to the outside of the tubular wall to form a coating, the coating covering the barrier and at least a portion of the tubular wall on at least one axial end of the barrier, and curing the coating to form the balloon.

Applying the elastomer solution may include dipping. According to an embodiment, the barrier includes ePTFE. Additionally or alternatively, the barrier remains in place after the coating is cured.

According to an embodiment, the method further includes cleaning the tubular element. The method may additionally include repeatedly applying the elastomer solution to approximately the same area to form multiple coating layers. According to an embodiment, the elastomer solution includes silicone, natural rubber, neoprene, nitrile, or a combination thereof. The elastomer solution may additionally or alternatively include one or more organic solvents.

In another aspect, the present disclosure relates to a method of using a medical device consistent with the medical devices described herein, the method including pushing air through the proximal end of the medical device to permeate the barrier and expand the balloon.

### BRIEF DESCRIPTION OF THE FIGURES

FIG. 1A is a partial schematic side view of a medical device including a balloon in a deflated configuration, consistent with the devices of this disclosure.

FIG. 1B is a partial schematic side view of the medical device of FIG. 1A, including a balloon in an inflated configuration according to an embodiment.

FIG. 1C is a partial schematic perspective view of the medical device of FIG. 1A, including a balloon in a deflated configuration according to an embodiment.

FIG. 1D is a partial schematic perspective view of the medical device of FIG. 1A, including a balloon in an inflated configuration according to an embodiment.

FIG. 2A is a partial schematic side view of a medical device including a balloon in a deflated configuration, consistent with the devices of this disclosure.

FIG. 2B is a partial schematic side view of the medical device of FIG. 2A, including a balloon in an inflated configuration according to an embodiment.

FIG. 3A is a partial schematic side view of a medical device including a balloon in a deflated configuration, consistent with the devices of this disclosure.

FIG. 3B is a partial schematic side view of the medical device of FIG. 3A, including a balloon in an inflated configuration according to an embodiment.

FIG. 4 is a flow diagram of a method of making the medical device shown in FIGS. 1A-3B consistent with the embodiments of this disclosure.

FIG. 5 is an image of the medical device with a balloon formed in Example 1.

FIGS. 6A-6C are images of the medical device with a balloon formed in Example 2.

The figures are rendered primarily for clarity and, as a result, are not necessarily drawn to scale. Moreover, various structure/components may be shown diagrammatically or removed from some or all of the views to better illustrate aspects of the depicted embodiments, or where inclusion of such structure/components is not necessary to an understanding of the various exemplary embodiments described herein. The lack of illustration/description of such structures/components in a particular figure is, however, not to be interpreted as limiting the scope of the various embodiments in any way.

### Definitions

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

As used herein, the term “elastomer” is used to refer to a material that is deformable. An elastomer typically can stretch considerably upon application of pressure or force and may return to approximately its original state upon release of said pressure or force. An elastomer is typically a polymer.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to”, “at most”, or “at least” a particular value, that value is included within the range. A range recited as a value “or greater” includes that particular value. A range recited as a value “or less” includes that particular value.

The term “about” is used here in conjunction with numeric values to include normal variations in measurements as expected by persons skilled in the art, and is understood have the same meaning as “approximately” and to cover a typical margin of error, such as ±5% of the stated value.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like. Furthermore, compositions of the present disclosure can be used to achieve methods of the present disclosure.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Reference in the specification to “some embodiments,” “an embodiment,” “one embodiment,” “embodiments,” “one or more embodiments,” or “other embodiments” means that a particular feature, structure, or characteristic described in connection with the embodiments is included in at least some embodiments, but not necessarily all embodiments, of the present disclosures.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

For any method disclosed herein that includes discrete steps, the steps may be conducted in any feasible order. And, as appropriate, any combination of two or more steps may be conducted simultaneously.

All headings throughout are for the convenience of the reader and should not be used to limit the meaning of the text that follows the heading, unless so specified.

### DETAILED DESCRIPTION

Balloons can be used in medical devices to provide temporary, reversible pressure or expansion. As one example, catheters including thin-walled balloons are frequently used during placement of stents. Manufacture of medical balloons as a component of a tubular structure, such as a lead or catheter, presents some technical challenges. During procedures using medical devices with balloons, it is desired that the balloon remain attached to the device and reliably inflate. To manufacture a balloon that is securely attached to the device, an elastomer solution is often cast directly onto the device (e.g., the tubular structure). The tubular structure is provided with air holes to inflate and deflate the balloon. For reliable performance, air holes may be installed in such a manner that they are not sealed by the balloon during inflation or deflation. It is desirable that the elastomer solution does not seal or clog the air holes during manufacturing. In prior art methods, the air holes on the tubular structure are closed, e.g., by using a meltable wax material, prior to casting to prevent clogging. Before the device can be used, the closing mechanism must be removed. Alternatively, the balloon may be manufactured separately from and attached to the tubular structure, however, this can present issues with the attachment of the balloon. It would be desirable to provide a method of making an inflatable balloon directly on a tubular structure using an air hole closing mechanism that does not need to be removed prior to use.

The present disclosure provides a method of making a medical device with an inflatable balloon. The present disclosure provides a method of directly forming a balloon on a tubular element by dipping the tubular element in an elastomer solution. The method includes using a barrier on the tubular element that does not need to be removed prior to use. This may advantageously streamline production of the balloon and reduce potential damage to the medical device associated with removal of a blocking material, such as a wax. The barrier may reduce or prevent clogging of the air holes on the tubular element by the elastomer solution. The barrier may reduce or prevent adhesion of the elastomer solution to the tubular element. The present disclosure further describes a medical device with an inflatable balloon and a barrier between a tubular element of the medical device and the inflatable balloon.

In one or more embodiments, the medical device is an implantable medical device. The medical device may be configured for use as a cardiac pacemaker, as a nerve stimulator, as a cochlear implant, as a urological implant, or otherwise as an implantable medical device. In one or more embodiments, the medical device is intended for temporary use. The medical device may be configured to be used during surgery or implantation of a second medical device, such as a stent. In one or more embodiments, the medical device is a catheter. The medical device may be configured for delivery or removal of fluids. In one or more embodiments, the medical device is a pacing lead. The medical device may include one or more electrodes, one or more fixation elements, and/or one or more controllers. In one or more embodiments, the medical device is attached to a proximal controller or proximal connector.

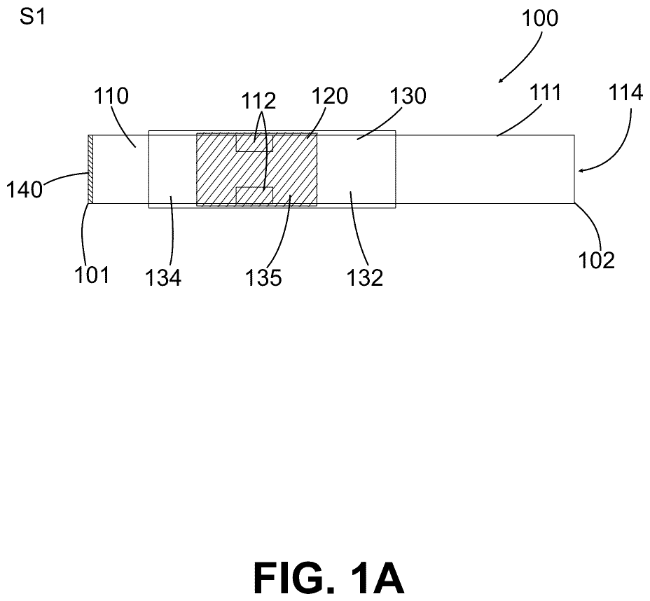

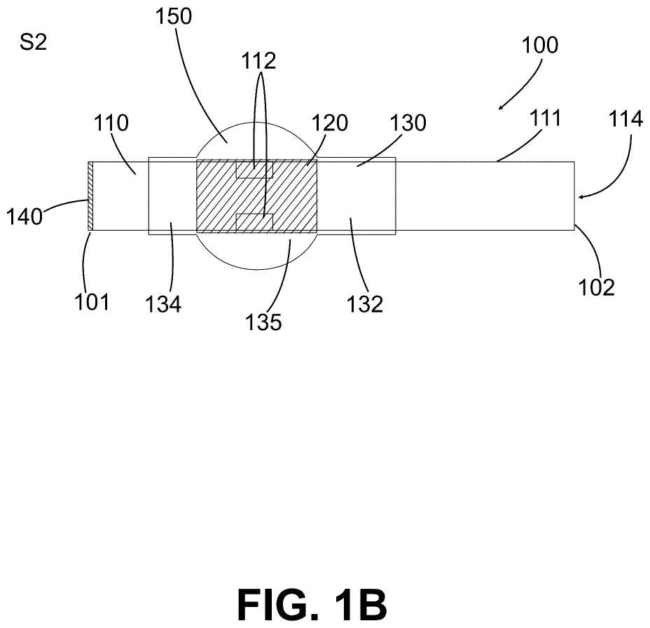

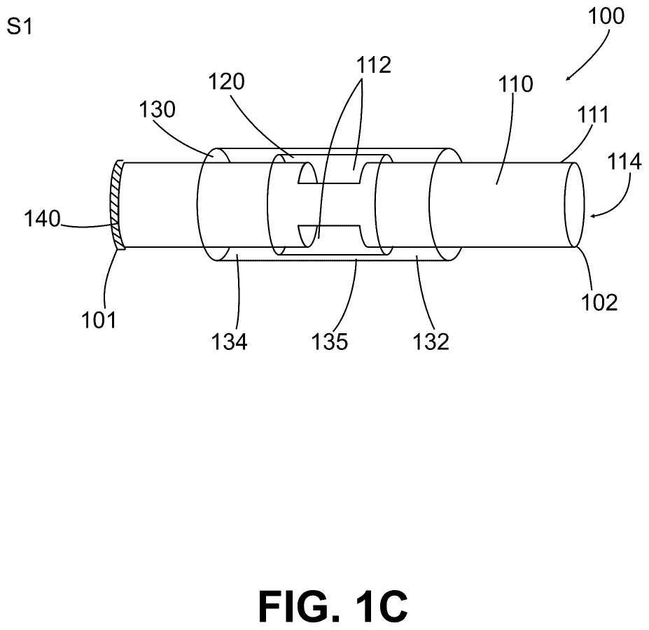

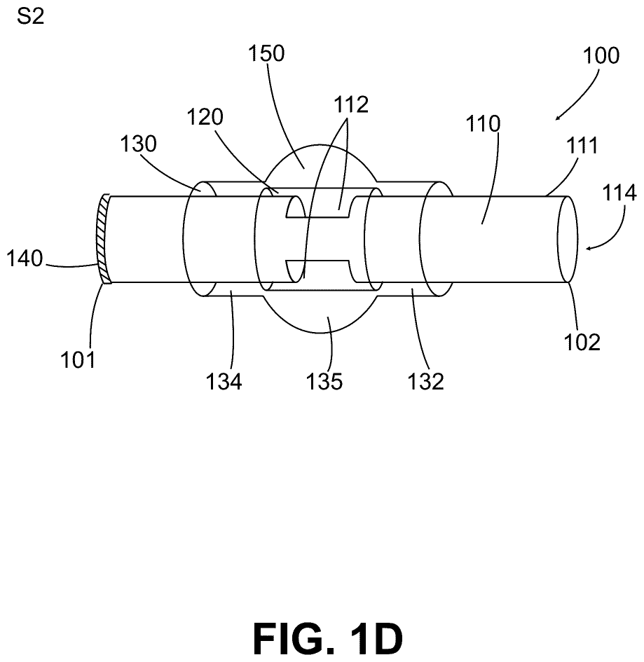

In one aspect, the present disclosure provides a medical device including a balloon. FIGS. 1A-1D show an example of a medical device 100 consistent with embodiments of this disclosure. The medical device 100 includes a tubular element 110 with a tubular wall 111. The tubular element 110 has a distal end 101 and a proximal end 102. One or more holes 112 extend through the tubular wall 111 between the distal end 101 and proximal end 102 of the tubular element 110. The medical device 100 also includes a balloon 130 extending diametrically around the tubular wall. FIGS. 1A and 1C show a schematic depiction of the medical device 100 with the balloon 130 in the deflated configuration S1. FIGS. 1B and 1D show a schematic depiction of the medical device 100 with the balloon 130 in the inflated configuration S2.

The balloon 130 is bonded to at least a portion of the tubular wall 111, and at least a portion of the balloon 130 is not bonded to the tubular wall 111. In the exemplary embodiment shown in FIGS. 1A-1D, the balloon 130 has a proximal portion 132 and a distal portion 134 that are bonded to the tubular wall 111. The balloon 130 has an inflatable middle portion 135 between the bonded proximal and distal portions 132, 134. According to an embodiment, the inflatable middle portion 135 is not bonded to the tubular wall 111. The inflatable middle portion 135 may be the segment which expands during inflation as shown by FIG. 1B. The medical device 100 further comprises a barrier 120 between the tubular wall 111 and the balloon 130. The barrier 120 covers the one or more holes 112 through the tubular wall 111 and is permeable to air. The inflatable middle portion 135 has an inner volume that is in fluid communication with the inner lumen 114 of the tubular element 110 through the barrier 120 and the one or more holes 112. Therefore, gas or liquid (e.g., air) pushed into the inner lumen 114 can be used to inflate the balloon 130.

The tubular element 110 is generally configured to allow the balloon 130 to be positioned in a desired location. The tubular element 110 typically has a distal end 101 and a proximal end 102. The inner lumen 114 of the tubular element extends between the two distal and proximal ends and is defined by the tubular wall. The inner lumen 114 of the tubular element may be empty, or it may hold additional elements, such as wires. In one or more embodiments, one end of the tubular element is sealed to be airtight. This may allow air to be pushed into the lumen of the tubular element, inflating the balloon. In the example shown in FIGS. 1A-1D, the distal end 101 is closed with a seal 140. The seal is generally configured to prevent air from flowing through the distal end 101. Any suitable method of sealing the tubular element may be used, such as applying adhesive, melting, crimping, or applying a physical stopper. The proximal end 102 may be connected to a controller or proximal connector.

The tubular wall 111 is generally configured to be a fluid-tight barrier between the external environment and the inner lumen 114 of the tubular element 110. The tubular wall 111 generally does not change shape when the balloon 130 is inflated. Thus, the material of the tubular element 110 is typically more rigid than the balloon 130. The tubular wall 111 may have any suitable thickness. In one or more embodiments, the tubular element 110 may be flexible to enable implantation in and/or navigation through the body.

The tubular element 110 may have any suitable length. In one or more embodiments, the tubular element 110 may have a length of 1 centimeter (cm) or more, 2 cm or more, 4 cm or more, 6 cm or more, 10 cm or more, 20 cm or more, 30 cm or more, 50 cm or more, 100 cm or more, or 200 cm or more. In one or more embodiments, the tubular wall may have a length of 500 cm or less, 300 cm or less, 100 cm or less, 80 cm or less, 60 cm or less, 40 cm or less, or 20 cm or less.

The tubular wall 111 may be formed of any material suitable for use in a medical device. In one or more embodiments, the tubular wall 111 may include or be made of polymer, glass, metal, or a combination thereof. In one or more embodiments where the tubular wall 111 includes a polymer, the polymer may be a thermoplastic, such as polyurethane, polyether block amide, CARBOTHANE™ (a polycarbonate-based thermoplastic polyurethane available from Lubrizol Corporation in Wickliffe, OH), PELLETHANE® (a thermoplastic polyurethane available from Lubrizol Corporation), or a combination thereof.

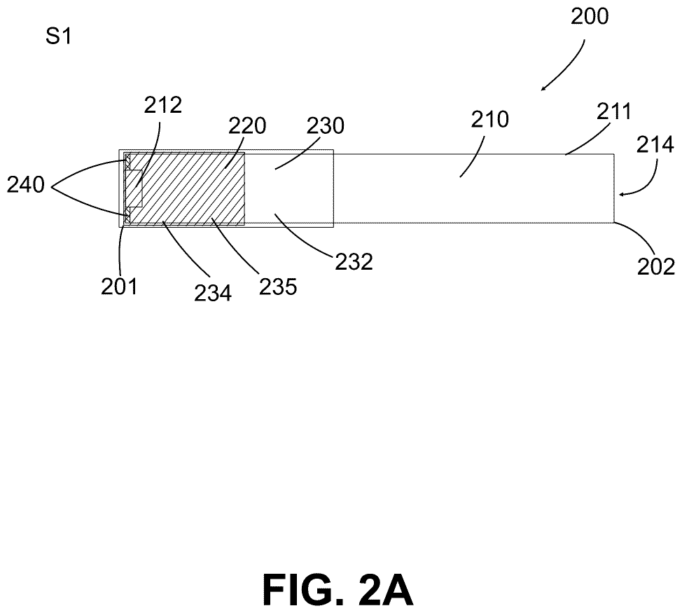

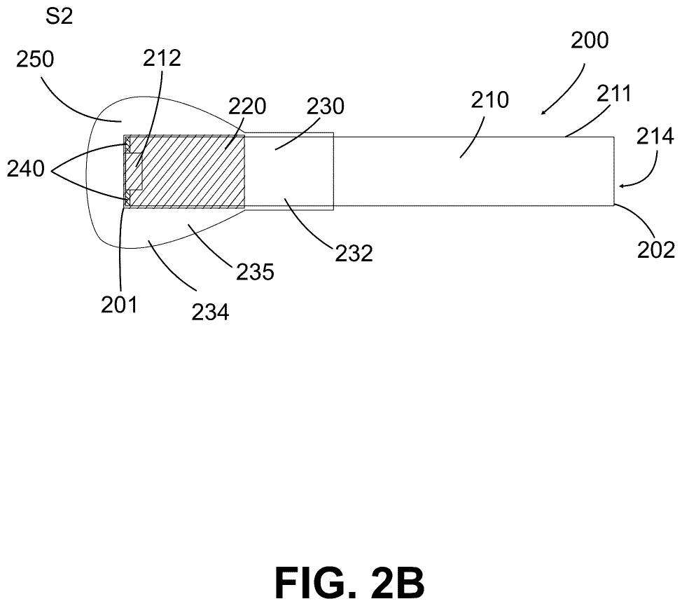

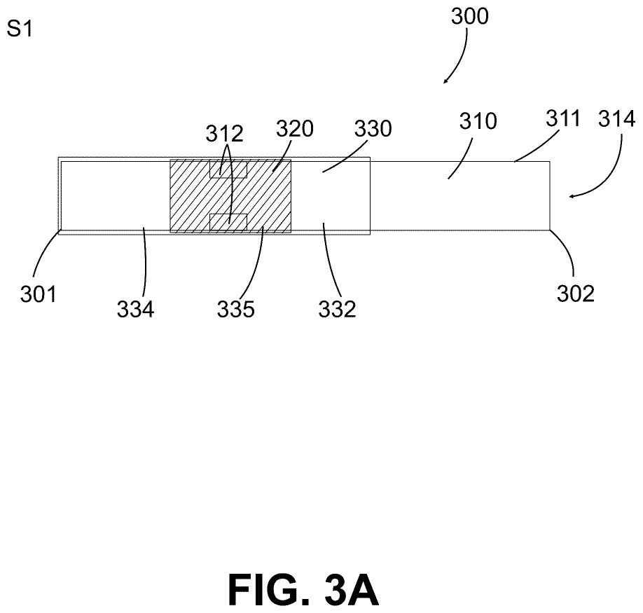

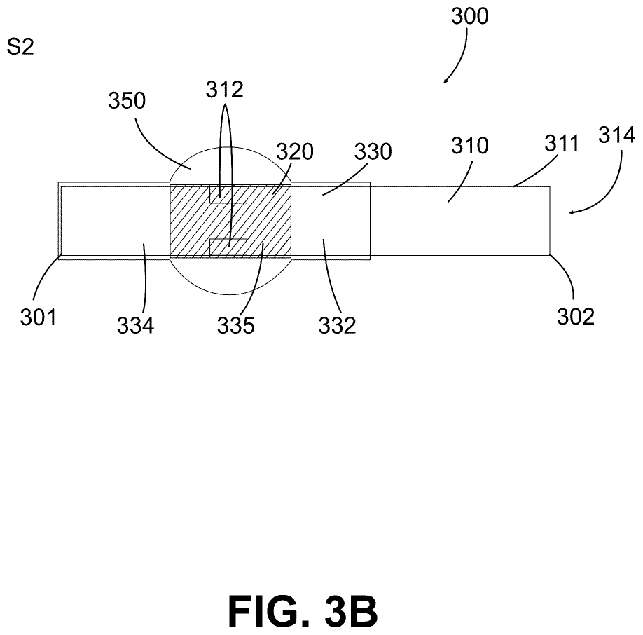

The one or more holes 112 through the tubular wall 111 are generally configured to allow air to pass from the inner lumen 114 of the tubular element 110 into the balloon 130. In one or more embodiments, including the example shown in FIGS. 2A and 2B, there may be one hole 212 extending through the tubular wall 211. In the example shown in FIGS. 2A and 2B, the one hole 212 is positioned on the distal end 201 of the tubular element 210. In some other embodiments, the distal end 201 of the tubular element may be covered by the balloon 230 to form a seal. FIGS. 3A and 3B show an example wherein the distal end 301 of the tubular element 310 is covered by the balloon 330, making it airtight.

In one or more other embodiments, including the examples shown in FIGS. 1A-1D and 3A-3B, there may be more than one hole 112 though the tubular wall. In one or more embodiments where there are multiple holes 112 through the tubular wall 111, the holes 112 may be arranged in any suitable manner. It may be desirable to arrange the holes 112 such that they are not plugged by the balloon 130 during deflation. It may be desirable to arrange the holes 112 such that air enters the balloon 130 at multiple locations during inflation. When air enters the balloon 130 at multiple locations, it may promote more distributed inflation. This may promote even wear of the balloon 130 over multiple uses. In one or more embodiments, the holes 112 may be arranged radially along an axial plane of the tubular element 110. In one or more embodiments, the holes 112 may be distributed along a portion of the tubular element 110.

The barrier 120 is generally configured to separate a portion of the balloon 130 from a portion of the tubular element 110. In one or more embodiments, the barrier 120 extends around the tubular element 110. According to an embodiment, the barrier 120 covers the entirety of the one or more holes 112. The barrier 120 may be applied onto the tubular element 110 by any suitable method. For example, the barrier 120 may be a piece of material wrapped around the tubular element 110. In one or more embodiments, the barrier 120 is attached to the tubular element 110. The barrier 120 may be closely fitted to the outside of the tubular element 110. In contrast to current medical devices with balloons, the barrier 120 of this disclosure may remain in place during inflation and deflation of the balloon 130. According to an embodiment, the barrier 120 is not removed from the medical device prior 100 to use, e.g., inflation or deflation of the balloon 130.

The barrier 120 is generally permeable to air. During inflation, air passes from the inner lumen 114 of the tubular element 110 through the hole(s) 112, though the barrier 120, and into the balloon 130. The barrier 120 is generally impermeable to the material of the balloon 130, including when the material of the balloon is in solution. For example, if the balloon 130 includes nitrile, the barrier 120 is typically impermeable to a solution of nitrile in a solvent. If the balloon 130 includes silicone, the barrier 120 is typically impermeable to a solution of silicone in a solvent.

The barrier 120 may be formed from any suitable material. Suitable materials are permeable to air and impermeable to the material of the balloon in solution (e.g., an elastomer solution). In one or more embodiments, the barrier 120 includes polytetrafluoroethylene (PTFE). In one or more embodiments, the barrier 120 includes expanded PTFE (ePTFE). The barrier 120 may be a tubular sleeve, or it may be a piece of material applied onto or wrapped around the tubular element 110.

The permeability of the barrier 120, in particular an ePTFE barrier, may be influenced by its density. It may also be influenced by parameters such as internodal distance, pore density, porosity, pore size, etc. Pore density may be measured using any suitable assay. One measure of pore density commonly used for materials such as those compatible with use as a barrier is standard density. Generally, higher pore density results in lower standard density. Thus, standard density may be used as a proxy for inverse pore density.

In one or more embodiments, the barrier is made of ePTFE having a standard density of 0.1 g/cm³ or less, 0.2 g/cm³ or less, 0.4 g/cm³ or less, 0.6 g/cm³ or less, 0.8 g/cm³ or less, 1.0 g/cm³ or less, 1.2 g/cm³ or less, or 1.5 g/cm³ or less. In one or more embodiments, the barrier has a standard density of 0.05 g/cm³ or more, 0.1 g/cm³ or more, 0.3 g/cm³ or more, 0.5 g/cm³ or more, 0.7 g/cm³ or more, 0.9 g/cm³ or more, or 1.0 g/cm³ or more.

Internodal distance, or the distance between pores, can be measured using light microscopy. According to an embodiment, the barrier 120 has an internodal distance of 5 micrometers (μm) or more, 10 μm or more, 15 μm or more, 20 μm or more, 25 μm or more, 30 μm or more, 40 μm or more, 50 μm or more, 60 μm or more, 80 μm or more, or 100 μm or more. In one or more embodiments, the barrier 120 has an internodal distance of 120 μm or less, 100 μm or less, 90 μm or less, 70 μm or less, 60 μm or less, or 40 μm or less

According to an embodiment, the barrier 120 has a wall thickness of 0.02 millimeters (mm) or more, 0.04 mm or more, 0.06 mm or more, 0.08 mm or more, 0.1 mm or more, 0.2 mm or more, 0.3 mm or more, 0.4 mm or more, 0.6 mm or more, or 1 mm or more. The barrier 120 may have a wall thickness of 2 mm or less, 1.5 mm or less, 1 mm or less, 0.7 mm or less, 0.5 mm or less, 0.4 mm or less, 0.3 mm or less, 0.1 mm or less, or 0.08 mm or less. The wall thickness of the barrier may range from 0.02 mm to 2 mm, from 0.1 mm to 1.5 mm, or from 0.2 mm to 1 mm.

In one or more embodiments, the medical device 100 comprises one balloon 130. In some other embodiments, the medical device 100 comprises more than one balloon 130, such as two or three balloons. Examples of medical devices that may include more than one balloon include catheters and dilators.

The balloon 130 is generally made of a material that can be repeatedly inflated and deflated. In one or more embodiments, the balloon 130 includes or is made of an elastomer. The elastomer may be a thermoset elastomer or a thermoplastic elastomer. Suitable elastomers include silicone, natural rubber (e.g., latex), polyurethane, or synthetic rubber, such as neoprene or nitrile. In one or more embodiments, the balloon 130 is made of silicone. It should be apparent to one of ordinary skill in the art that any suitable elastic material may be used.

In embodiments wherein the balloon 130 is made of a polymer, the polymer may have any suitable hardness. Hardness may be measured, for example, using a Shore durometer. Shore hardness is typically reported as either an “A” or a “D” hardness. Measurement of hardness using the Shore durometer is described in greater detail in U.S. Pat. No. 1,770,045, published on Jul. 8, 1930. Polymers suitable for use in the elastomer solution typically have a hardness of 40 A or more, 50 A or more, 60 A or more, 70 A or more, 80 A or more, or 90 A or more. Polymers suitable for use in the elastomer solution typically have a hardness of 100 A or less, 95 A or less, or 85 A or less. The polymer may have a hardness in the range of 65 A to 95 A or from 70 A to 90 A, or about 80 A.

As shown in FIGS. 1A-1D, the balloon 130 generally extends over the barrier 120 and the one or more holes 112. In one or more embodiments, including the example shown in FIGS. 1A-1D, the balloon 130 is bonded to the tubular wall 111 adjacent to the distal and proximal ends 101, 102 of the barrier 120. That is, both the proximal portion 132 and the distal portion 134 of the balloon 130 are bonded to the tubular wall 111. The inflatable middle portion 135 is not bonded to the tubular wall 111. As shown in FIGS. 1B and 1D, the inflatable middle portion 135 of the balloon 130 that is positioned over the barrier 120 expands and inflates when air is pushed into to the tubular element 110. This results in an inflated portion 150 of the balloon 130. The proximal portion 132 and the distal portion 134 of the balloon 130 that are not positioned over the barrier remain affixed (e.g., bonded) to the tubular element 110 when air is pushed into the tubular element 110.

In an alternative embodiment of the medical device 200 shown in FIGS. 2A and 2B, the balloon 230 is positioned at the distal end 201 of the tubular wall 211. The proximal portion 232 of the balloon is bonded to the tubular wall 211. That is, the balloon 230 may be bonded to the tubular wall 211 adjacent only to the proximal end of the barrier 220. The distal portion 234 of the balloon 230 forms the inflatable portion 235. The distal end 201 of the tubular element 210 may be at least partially sealed by seal 240. One hole 212 is disposed at or near the distal end 201. A barrier 220 is disposed about the distal end, covering the hole 212. When air is pushed through the inner lumen 214 of the tubular element 210 and through the hole 212 and the barrier 220, the inflatable portion 235 inflates, forming the inflated portion 250. FIG. 2A shows a schematic depiction of the medical device 200 with the balloon 230 in the deflated configuration S1. FIG. 2B shows a schematic depiction of the medical device 200 with the balloon 230 in the inflated configuration S2.

In another alternative embodiment of the medical device 300, shown in FIGS. 3A and 3B, the distal end 301 of the tubular wall 311 is covered by the balloon 330. However, the one or more holes 312 are positioned along the tubular wall 311 (similar to the embodiment of FIGS. 1A-1D) rather than at the distal end 301. The barrier 320 is disposed about the tubular wall 311 in the area of the one or more holes 312 such that the barrier 320 covers the one or more holes 312. The balloon 330 covers the distal end 301 of the tubular wall 311 and the barrier 320. The proximal portion 332 of the balloon 330 is bonded to the tubular wall 311. The distal portion 324 of the balloon 330 may also be bonded to the tubular wall 311. Alternatively, the distal portion 324 is not bonded to the tubular wall 311. The inflatable middle portion 325 of the balloon 330 is formed over the barrier 320 and may be inflated by pushing air through the inner lumen 314 of the tubular element 310 and through the one or more holes 312 and the barrier 320. FIG. 3A shows a schematic depiction of the medical device 300 with the balloon 330 in the deflated configuration S1. FIG. 3B shows a schematic depiction of the medical device 300 with the balloon 330 in the inflated configuration S2.

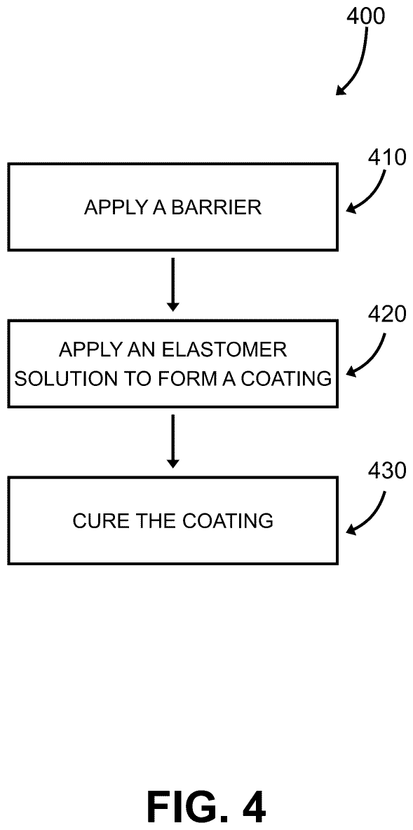

In another aspect, the present disclosure relates to a method of forming a medical device 100, 200, 300 including a balloon 130, 230, 330. A diagram of one example of this method is shown in FIG. 4. The method 400 includes a first step 410 of applying a barrier 120, 220, 320 to at least a portion of a tubular element 110, 210, 310. The tubular element 110, 210, 310 includes a tubular wall 111, 211, 311 having a distal end 101, 201, 301 and a proximal end 102, 202, 302, and one or more holes 112, 212, 312 through the tubular wall 111, 211, 311 between the distal and proximal ends. The barrier 120, 220, 320 is applied to the outside of the tubular element 110, 210, 310 and covers the one or more holes 112, 212, 312 that extend through the tubular wall 111, 211, 311. The barrier 120, 220, 320 is permeable to air. The method further includes a second step 420 of applying an elastomer solution to the outside of the tubular wall 111, 211, 311 to form a coating. The elastomer solution may be applied, for example, by dipping the end of the tubular wall 111, 211, 311 into the elastomer solution. Other methods of applying may also be used, such as spray coating, brushing, etc. The coating covers the barrier 120, 220, 320 and at least a portion of the tubular wall 111, 211, 311 adjacent the barrier 120, 220, 320. The coating covers at least one axial end of the barrier 120, 220, 320. The coating may also cover the distal end of the tubular wall 111, 211, 311. The method then includes a third step 430 of curing the coating to form a balloon 130, 230, 330.

The barrier 120, 220, 320 is generally consistent with the materials and properties of barriers described herein. In one or more embodiments, the barrier includes ePTFE. The barrier is generally permeable to air and impermeable to the elastomer solution used to prepare the coating. The barrier 120, 220, 320 is configured to prevent the elastomer solution from blocking the one or more holes 112, 212, 312 that extend through the tubular wall while the elastomer solution is applied onto the tubular element 110, 210, 310. It may be desirable for the elastomer solution to not enter the inner lumen 114, 214, 314 of the tubular element 110, 210, 310.

In one or more embodiments, applying the barrier 120, 220, 320 includes wrapping a strip of material around the tubular element 110, 210, 310. In some other embodiments, applying the barrier includes positioning a tube of material around the tubular element 110, 210, 310. In one or more embodiments, the method further includes bonding the barrier 120, 220, 320 to the tubular element 110, 210, 310. It may be desirable to bond or otherwise affix the barrier 120, 220, 320 to the tubular element 110, 210, 310 to prevent the barrier 120, 220, 320 from moving during later steps of the method.

In one or more embodiments, the method further comprises cleaning the tubular element 110, 210, 310. This step may occur at any suitable point in the method. Cleaning the tubular element after the barrier is applied but before the elastomer solution is applied may improve bonding of the elastomer solution to the tubular element. In one or more embodiments, cleaning the tubular element includes plasma cleaning and/or siloxane cleaning.

Applying the elastomer solution to the tubular element 110, 210, 310 generally includes applying a thin layer of elastomer solution over the barrier 120, 220, 320 and at least a portion of the tubular element 110, 210, 310 not covered by the barrier 120, 220, 320. In one or more embodiments, applying the elastomer solution includes dipping the tubular element 110, 210, 310 into the elastomer solution. In one or more embodiments, applying the elastomer solution includes brushing or painting on a coating of the elastomer solution. As described herein, the elastomer solution generally covers the entirety of the barrier 120, 220, 320 and at least a portion of the tubular element 110, 210, 310 not covered by the barrier 120, 220, 320. The elastomer solution may be applied to the tubular element 110, 210, 310 adjacent to one or both axial ends of the barrier 120, 220, 320. The elastomer solution is generally applied in a way to prevent the elastomer solution from entering the inner lumen 114, 214, 314 of the tubular element 110, 210, 310. The viscosity of the elastomer solution may be adjusted by changing the volume of solvent used. The thickness of the layer of elastomer solution applied may be impacted by the viscosity of the elastomer solution.

The elastomer solution may include any suitable components as described herein. In one or more embodiments, the elastomer solution includes an elastomer dissolved or suspended in a solvent. In one or more embodiments, the elastomer includes a polymer. The elastomer may include silicone, polyurethane, natural rubber (e.g., latex), synthetic rubber such as neoprene or nitrile, or a combination thereof. The polymer may have any suitable hardness as described herein. In one or more embodiments, the elastomer includes a monomer.

The elastomer may be a liquid elastomer, or it may be a solid elastomer. The elastomer may be cured after application onto the tubular element. In some embodiments, the elastomer may be partially cured prior to application to reduce curing time and/or temperature to cure the elastomer after application. In one or more embodiments, the solvent may include an aqueous solvent, such as water. In one or more embodiments, the solvent may include one or more organic solvents. The organic solvent may include hexane, heptane, tetrahydrofuran, dimethylacetamide, and other non-polar solvents. In one or more embodiments, the elastomer solution is a low durometer polyurethane solution in an organic solvent, such as tetrahydrofuran or dimethylacetamide.

In one or more embodiments, one or more additional layers of elastomer solution may be applied to the tubular element 110, 210, 310. An additional layer of elastomer solution may be applied before the previous layer is cured, when the previous layer is partially cured, or when the previous layer is fully cured. In one or more embodiments, at least 1, at least 2, at least 3, at least 4, at least 5, or at least 6 layers of elastomer solution are applied to the tubular element 110, 210, 310. In one or more embodiments, at most 10, at most 8, at most 7, at most 6, at most 5, at most 4, at most 3, or at most 2 layers of elastomer solution are applied to the tubular element 110, 210, 310. In embodiments where multiple layers are applied, each layer may be applied to approximately the same area of the tubular element 110, 210, 310 such that the layers partially or completely overlap.

Curing the elastomer solution generally includes curing the elastomer solution so that it is solid and no longer reactive. The curing conditions may depend on the type of elastomer and solvent used to prepare the coating. In one or more embodiments, curing the elastomer solution includes drying. Drying may occur at ambient temperature, or it may occur at an elevated temperature. As used here, “ambient temperature” refers to the temperature of the manufacturing space, typically between 20° C. and 30° C. In one or more embodiments, curing the elastomer solution includes applying energy, such as ultraviolet radiation, to the coating. The elastomer solution may be cured for any suitable amount of time sufficient to cure the elastomer. For example, the curing time may be as short as a few minutes, to as long as several days. In one or more embodiments, the elastomer solution is cured for 1 hour to 72 hours, from 2 hours to 48 hours, or from 4 hours to 24 hours.

According to an embodiment, the method of making the medical device does not include removing the barrier from the device. That is, the barrier remains a part of the medical device, disposed between the tubular element and the balloon.

In another aspect, the present disclosure relates to a method of using a medical device 100, 200, 300 including a balloon 130, 230, 330. The method may be applicable to any medical device including a balloon described herein. The method includes pushing air through the proximal end to permeate the barrier 120, 220, 320 and expand the balloon 130, 230, 330. This produces an inflated portion 150 of the balloon 130. In FIGS. 1A-3B, the deflated state of the balloon is labeled as “S1” and the inflated state of the balloon is labeled as “S2”. An example of the inflated portion 150, 250, 350 is shown in FIGS. 1B, 1D, 2B, and 3B. As shown in FIG. 1B, the distal end 101 of the tubular element is closed with a seal 140 and air is pushed into the tubular element through the proximal end 102. The balloon 130 is attached to the tubular element 110 on both axial ends of the barrier 120. When air is pushed through the proximal end 102, the air flows through the one or more holes 112 and through the barrier 120. Resultantly, the inflatable middle portion 135 (the portion of the balloon 130 covering the barrier 120) inflates to form a medical device including a balloon with an inflated portion 150 as shown in FIGS. 1B and 1D.

According to an embodiment, the method of using the medical device does not include removing the barrier from the device. That is, the barrier remains a part of the medical device during use, disposed between the tubular element and the balloon.

Different shapes of inflated balloons may be result from different methods of balloon production. FIG. 2B shows an example of a medical device 200 with a balloon 230 where the balloon 230 is bonded to the tubular element 210 adjacent the distal end 201 of the tubular element 210. The distal end 201 is sealed with a seal 240 and one hole 212 is present at or near the distal end 201. The hole 212 is covered by the barrier 220. When air is pushed into the proximal end 202, it passes through the one or more holes 212 and the barrier 220 and into the balloon 230. The shape of the inflated portion 250 of FIG. 2B is different from the shape of the inflated portion 150 of FIG. 1B due to its placement at the tip of the tubular element 210. Each of these shapes may provide certain advantages in different applications.

### EXEMPLARY EMBODIMENTS

Embodiment 1 is a medical device comprising a balloon, the medical device comprising: a tubular element comprising a tubular wall comprising a distal end and a proximal end, the tubular element comprising one or more holes through the tubular wall between the distal and proximal ends;

a balloon extending diametrically around the tubular wall, wherein the balloon is bonded to at least a portion of the tubular wall, and wherein at least a portion of the balloon is not bonded to the tubular wall; and a barrier between the tubular wall and the balloon, the barrier covering the one or more holes and being permeable to air.

Embodiment 2 is the medical device of embodiment 1, wherein the tubular wall comprises polymer, glass, or metal.

Embodiment 3 is the medical device of embodiment 2, wherein the polymer comprises a thermoplastic.

Embodiment 4 is the medical device of embodiment 3, wherein the thermoplastic comprises polyurethane, polyether block amide, CARBOTHANE, or PELLETHANE.

Embodiment 5 is the medical device of any of embodiments 1 to 4, wherein the medical device comprises a inner lumen.

Embodiment 6 is the medical device of any of embodiments 1 to 5, wherein one axial end of the tubular element is sealed.

Embodiment 7 is the medical device of any of embodiments 1 to 6, wherein the medical device comprises a lead.

Embodiment 8 is the medical device of embodiment 7, wherein the lead comprises one or more electrodes.

Embodiment 9 is the medical device of any of embodiments 7 or 8, wherein the lead comprises a fixation element.

Embodiment 10 is the medical device of any of embodiments 1 to 9, wherein the medical device comprises a catheter.

Embodiment 11 is the medical device of any of embodiments 1 to 10, wherein the tubular element comprises more than one hole through the tubular wall.

Embodiment 12 is the medical device of embodiment 11, wherein the holes are arranged radially along an axial plane of the tubular element.

Embodiment 13 is the medical device of any of embodiments 1 to 12, wherein the balloon comprises an elastomer.

Embodiment 14 is the medical device of embodiment 13, wherein the elastomer is a thermoset elastomer.

Embodiment 15 is the medical device of embodiment 13, wherein the elastomer is a thermoplastic elastomer.

Embodiment 16 is the medical device of embodiment 13, wherein the elastomer comprises silicone, natural rubber (e.g., latex), synthetic rubber, such as neoprene or nitrile.

Embodiment 17 is the medical device of any of embodiments 1 to 16, wherein the balloon is bonded to the tubular wall adjacent to both axial ends of the barrier.

Embodiment 18 is the medical device of any of embodiments 1 to 17, wherein the barrier has a density of 1.0 g/cm³ or less.

Embodiment 19 is the medical device of any of embodiments 1 to 18, wherein the barrier has a wall thickness of 0.2 mm or less.

Embodiment 20 is the medical device of any of embodiments 1 to 19, wherein the barrier comprises polytetrafluoroethylene (PTFE).

Embodiment 21 is the medical device of embodiment 20, wherein the PTFE comprises expanded PTFE (ePTFE).

Embodiment 22 is a method of forming a medical device comprising a balloon, the method comprising:
applying a barrier to at least a portion of a tubular element, the tubular element comprising a tubular wall having a distal end and a proximal end and one or more holes through the tubular wall between the distal and proximal ends, the barrier covering the one or more holes and being permeable to air;
applying an elastomer solution to the outside of the tubular wall to form a coating, the coating covering the barrier and at least a portion of the tubular wall on at least one axial end of the barrier; and
curing the coating to form the balloon.

Embodiment 23 is the method of embodiment 22, wherein the barrier comprises ePTFE.

Embodiment 24 is the method of embodiments 22 or 23, wherein applying the barrier comprises wrapping a strip of material around the tubular element.

Embodiment 25 is the method of any of embodiments 22 to 24, further comprising bonding the barrier to the tubular element.

Embodiment 26 is the method of any of embodiments 22 to 25, wherein the barrier remains in place after the coating is cured.

Embodiment 27 is the method of any of embodiments 22 to 26, further comprising cleaning the tubular element.

Embodiment 28 is the method of embodiment 27, wherein cleaning comprises plasma cleaning or siloxane removal.

Embodiment 29 is the method of embodiments 22 to 28, further comprising repeatedly applying the elastomer solution to approximately the same area to form multiple coating layers.

Embodiment 30 is the method of embodiment 29, wherein a total of three coating layers are applied.

Embodiment 31 is the method of any of embodiments 22 to 30, wherein applying the elastomer solution comprises dipping.

Embodiment 32 is the method of any of embodiments 22 to 31, wherein the elastomer solution comprises silicone, natural rubber (e.g., latex), synthetic rubber, such as neoprene or nitrile.

Embodiment 33 is the method of any of embodiments 22 to 32, wherein the elastomer solution comprises one or more organic solvents.

Embodiment 34 is the method of any of embodiments 22 to 33, wherein the elastomer solution is cured for at least 24 hours.

Embodiment 35 is a method of using the medical device of any of embodiments 1 to 21, the method comprising pushing air through the proximal end to permeate the barrier and expand the balloon.

### EXAMPLES

| Reagent name | Source | Description |
|---|---|---|
| Polyurethane tube | Donatelle (New Bright, | 75D PELLETHANE - 0.053″ outer diameter |
| | MN) | (OD), 0.033″ inner diameter (ID) |
| Steel tube | | 0.083″ OD Steel Hypo-tube - 4″ length |
| 0.006″ wall ePTFE | Zeus (Orangeburg, | AEOS ePTFE; 0.09 +/− 0.003″ ID; 0.006 +/− |
| | South Carolina) | 0.002″ wall thickness; 0.65 +/− 0.15 g/cm³ |
| | OPN 248996 | density |
| 0.012″ wall ePTFE | Zeus | AEOS ePTFE; 0.083 +/− 0.003″ ID; 0.012 +/− |
| | OPN 179978 | .001″ wall thickness; 0.35 +/− 0.15 g/cm³ |
| | | density; |
| 0.0022″ wall ePTFE | Zeus | AEOS ePTFE; 0.099 +/− 0.005″ ID; 0.0022 +/− |
| | OPN 264444 | 0.0005″ wall thickness |
| MED1137 Silicone | Nusil (Carpinteria, | 1137 Medical Adhesive |
| | California) | |
| N-Heptane | e.g., Sigma Aldrich | |
| Latex solution | Holden's Latex | Pre-vulcanized casting latex, low viscosity |
| | (Macungie, | |
| | Pennsylvania,) | |
| | HX-200 | |

### Example 1

In this example, a medical device with a balloon was formed by dip coating.

A thin-walled polyurethane tube was obtained. Two holes were cut into the wall of the tube 180 degrees radially apart in the mid-section of the tubular element. A strip of 0.006″ (approximately 0.15 mm) wall ePTFE was placed radially around the holes, completely covering each hole. The strip of ePTFE was tightly wrapped around the tube. The tube with ePTFE was plasma cleaned for 10 minutes. The cleaned tube was then dipped into a solution of MED1137 silicone diluted 1:2 with N-heptane. The tube was dipped twice more for a total of three coats. The silicone solution was dried for 24 hours.

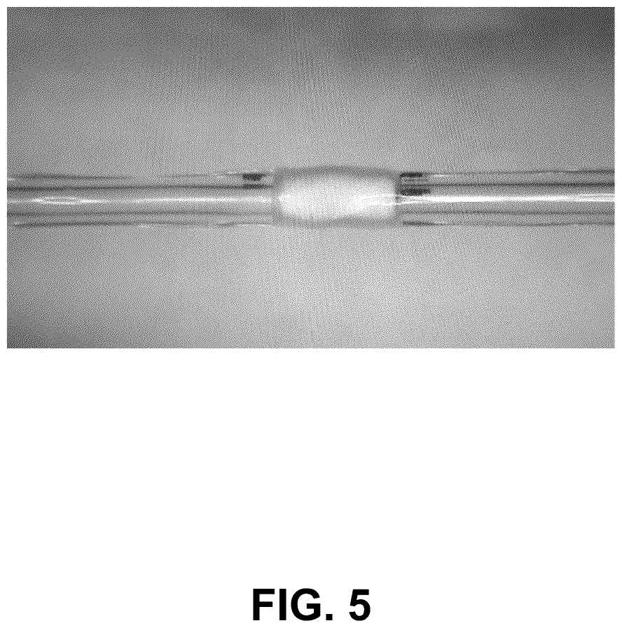

After the silicone had dried, one end of the tube was blocked and the other was attached to an inflation syringe. The balloon was inflated and observed to release from the ePTFE strip but stay bonded to the tube. The balloon formed is shown in FIG. 5.

From this example, it was learned that ePTFE can be used as a barrier in manufacture of a medical device including a silicone balloon.

### Example 2

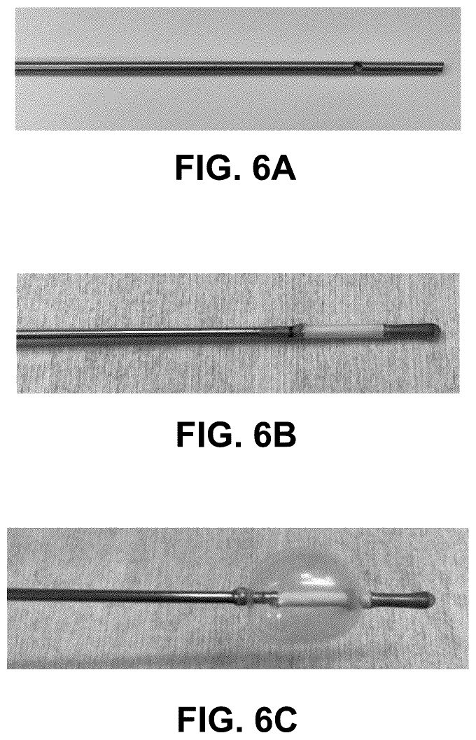

A steel tube with a length of 4 inches was obtained. A slot for inflation was cut into the tube using a circular saw. An example of a steel tube with an inflation slot is shown in FIG. 6A. A tube of 0.012″ (approximately 0.30 mm) wall ePTFE with a length of approximately 1.3 cm was slid onto the tube, covering the inflation slot. Each end of the ePTFE tube was bonded to the steel tube using a UV-cured glue. The tube was dipped into the latex solution and slowly removed to form an even coating. The coat was dried for 20 minutes, then was dipped again to form a second coating layer. The coatings were dried for 24 hours. An example of a steel tube with a dried latex coating is shown in FIG. 6B.

A needle was inserted into one end of the steel tube and used to inject approximately 2 cm³ of air to inflate the balloon. The latex released partially from the ePTFE liner before expanding radially. An example of a steel tube with an inflated latex balloon is shown in FIG. 6C.

From this Example, it was learned that a 0.012″ wall ePTFE tube allowed partial expansion of a latex balloon.

### Example 3

A latex balloon was prepared on a steel tube as described in Example 2. A tube of 0.0022″(approximately 0.056 mm) wall ePTFE was used as a barrier instead of a tube of 0.012″ wall ePTFE. The balloon was inflated using a needle with 2 cm³ of air as in Example 2. The latex released partially from the ePTFE liner before expanding radially.

From this Example, it was learned that a 0.0022″ wall ePTFE tube allowed partial expansion of a latex balloon.

### Example 4

A latex balloon was prepared on a steel tube as described in Example 2. A tube of 0.006″ (approximately 0.15 mm) wall ePTFE was used as a barrier instead of a tube of 0.012″ wall ePTFE. The balloon was inflated using a needle with 2 cm³ of air as in Example 2. The latex released acceptably from the ePTFE liner and inflated acceptably.

From this Example, it was learned that an ePTFE liner with a 0.006″ wall allowed acceptable inflation of a latex balloon. With the results of Examples 2 and 3, it was learned that ePTFE liners with a wall of at most 0.006″ performed better than ePTFE liners with thicker walls.

This application is intended to cover any adaptations or variations of the specific embodiments discussed herein. Therefore, it is intended that this disclosure be limited only by the claims and the equivalents thereof. The disclosed embodiments are presented for purposes of illustration and not limitation and although specific embodiments have been illustrated and described herein, it will be appreciated by those of ordinary skill in the art that a variety of alternate and/or equivalent implementations can be substituted for the specific embodiments shown and described without departing from the scope of the present disclosure.

What is claimed:

1. A medical device comprising:
a tubular element comprising a tubular wall with a distal end and a proximal end, the tubular element comprising an inner lumen and one or more holes through the tubular wall between the distal and proximal ends;
a balloon extending diametrically around the tubular wall, wherein the balloon is bonded to at least a portion of the tubular wall, and wherein at least a portion of the balloon is not bonded to the tubular wall; and
a barrier between the tubular wall and the balloon, the barrier covering the one or more holes and being permeable to air.

2. The medical device of claim 1, wherein one axial end of the tubular element is sealed.

3. The medical device of claim 1, wherein the tubular element comprises more than one hole through the tubular wall.

4. The medical device of claim 1, wherein the tubular wall comprises polymer, glass, or metal.

5. The medical device of claim 1, wherein the balloon comprises an elastomer.

6. The medical device of claim 5, wherein the elastomer comprises silicone, natural rubber, neoprene, nitrile, or a combination thereof.

7. The medical device of claim 1, wherein the balloon is bonded to the tubular wall adjacent to both axial ends of the barrier.

8. The medical device of claim 1, wherein the barrier has a density of 1.0 g/cm³ or less.

9. The medical device of claim 1, wherein the barrier has a wall thickness of 0.2 mm or less.

10. The medical device of claim 1, wherein the barrier comprises polytetrafluoroethylene (PTFE).

11. The medical device of claim 10, wherein the PTFE comprises expanded PTFE (ePTFE).

12. A method of forming a medical device comprising a balloon, the method comprising:
applying a barrier to at least a portion of a tubular element, the tubular element comprising a tubular wall having a distal end and a proximal end and one or more holes through the tubular wall between the distal and proximal ends, the barrier covering the one or more holes and being permeable to air;
applying an elastomer solution to the outside of the tubular wall to form a coating, the coating covering the barrier and at least a portion of the tubular wall on at least one axial end of the barrier; and
curing the coating to form the balloon.

13. The method of claim 12, wherein applying the elastomer solution comprises dipping.

14. The method of claim 12, wherein the barrier comprises ePTFE.

15. The method of claim 12, wherein the barrier remains in place after the coating is cured.

16. The method of claim 12, further comprising cleaning the tubular element.

17. The method of claim 12, further comprising repeatedly applying the elastomer solution to approximately the same area to form multiple coating layers.

18. The method of claim 12, wherein the elastomer solution comprises silicone, natural rubber, neoprene, nitrile, or a combination thereof.

19. The method of claim 12, wherein the elastomer solution comprises one or more organic solvents.

20. A method of using the medical device of claim 1, the method comprising
pushing air through the proximal end to permeate the barrier and expand the balloon.
